(19) United States
(12) Patent Application Publication
(10) Pub. No.: US 2013/0158603 A1
(43) Pub. Date: Jun. 20, 2013

(54) INTERSPINOUS PROCESS SPACER

(71) Applicants: Todd Bjork, River Falls, WI (US); Dan McPhillips, Ham Lake, MN (US); Garrett Ganske, Lino Lakes, MN (US); Dwight Tyndall, Flossmoor, IL (US); Kari Ellen Riina, Fishers, IN (US)

(72) Inventors: Todd Bjork, River Falls, WI (US); Dan McPhillips, Ham Lake, MN (US); Garrett Ganske, Lino Lakes, MN (US); Dwight Tyndall, Flossmoor, IL (US); Joseph Riina, Fishers, IN (US)

(21) Appl. No.: 13/572,316

(22) Filed: Aug. 10, 2012

Related U.S. Application Data
(60) Provisional application No. 61/522,663, filed on Aug. 11, 2011.

(51) Int. Cl. A61B 17/70

(52) U.S. Cl. CPC A61B 17/7062
USPC 606/249

(57) ABSTRACT

An interspinous process spacer is disclosed. The interspinous process spacer may be configured to be minimally invasive and adjustable.

### PRIORITY

This application claims the benefit of U.S. Provisional Application Ser. No. 61/522,663, filed on Aug. 11, 2011, which is hereby incorporated by reference in its entirety herein.

### FIELD

The present invention generally relates to an interspinous process spacer. More particularly, the present invention relates to a minimally invasive, adjustable interspinous process spacer.

### BACKGROUND

Lumbar spinal stenosis (“LSS”, and sometimes called sciatica) is a condition of the spine characterized by a narrowing of the lumbar spinal canal. With lumbar spinal stenosis, the spinal canal narrows and pinches the spinal cord and nerves, causing pain in the back and legs. The most common symptoms of spinal stenosis are pain and difficulty when walking, although numbness, tingling, hot or cold feelings in the legs, and weakness or tiredness may also be experienced. In extreme cases, spinal stenosis can cause cauda equina syndrome, a syndrome characterized by neuromuscular dysfunction that may result in permanent nerve damage.

A surgical technique has been developed in which the vertebrae are distracted and an interspinous process spacer is implanted to maintain the desired separation between the segments. This technique is somewhat less invasive than decompressive laminectomy, which may provide significant benefits to patients experiencing LSS symptoms.

As with other surgeries, when performing surgery to implant an interspinous process spacer, one consideration is the size of the incision that is required to allow introduction of the device. Medical treatments that can be performed in a less invasive manner are greatly sought after by the medical community and patients alike. In some procedures, less invasive techniques are advantageous because they have shorter recovery periods, result in little to no blood loss, and greatly decrease the chances of significant complications. Moreover, less invasive techniques are generally less expensive for the patient.

In view of the many advantages of less invasive procedures, it would be highly advantageous to have an interspinous process spacer and an associated procedure amenable to less invasive techniques.

### SUMMARY

An interspinous process spacer addressing the above-noted issues is disclosed. The interspinous process spacer may be configured to be minimally invasive and adjustable.

In an embodiment of the present invention, the interspinous process spacer may include a spacer body and bone contacting extensions. According to one aspect of the present invention, the bone contacting extensions may include bone engaging protrusions. In one embodiment, the bone engaging protrusions may be angled down toward the bone in the range of about 2-8 degrees to pull the implant down tightly to the bone.

In one embodiment of the present invention, the spacer may be implanted in as a one piece construct. In another embodiment, the spacer of the present invention may be assembled in situ.

In one embodiment of the present invention, the bone contacting extensions may be removably connected to a distraction tool. According to one aspect of the present invention, the spacer may be opened after implantation using a distraction tool. In an embodiment, the spacer of the present invention may be incrementally opened. In yet another embodiment, the movable bone contacting extensions may move independently to accommodate variations in patient's anatomy. In an embodiment, a screw or other mechanism may be used to tighten the bone contacting extensions into a desired position after distraction.

In yet another embodiment of the present invention, a spring clip or other mechanism may be connected to the distraction tool such that the distraction tool remains in an upright and tense position relative to the patient. In an embodiment of the present invention, the distraction tool is configured to keep the bone contacting extensions parallel to one another and aligned to the spacer body.

According to one embodiment of the present invention, the distraction tool may include markings to indicate the size of spacer needed for the particular patient anatomy. In yet another embodiment, a sizing tool may be used to determine what size spacer will fit in the patient's anatomy.

According to one embodiment of the present invention, the interior aspect of the bone contacting extensions may be contoured to allow for a tighter and closer fit down to the lamina. In an embodiment of the present invention, the exterior aspect of the bone contacting extensions may be configured to allow one spacer of the present to be placed directly abutting another spacer of the present invention to treat multiple spinal levels.

In an embodiment of the present invention, a male-female dovetail may be used to fit an implantation tool to the spacer.

In yet another embodiment of the present invention, a spacer sized smaller than the interspinous process space may be used with an anterior plate to compress the interspinous process space. In one embodiment of the present invention, the spacer may include dimples or other features to interact with a compression tool. According to one aspect of the present invention, a screw or other mechanism may be used to tighten the bone contacting extensions into a desired position after compression.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 depicts a disassembled view of a spacer according to an embodiment of the present invention.

FIG. 2 is a perspective view of an embodiment of the present invention.

FIG. 3 is a perspective view of an embodiment of the present invention.

FIG. 4 is a perspective view of an embodiment of the present invention.

FIG. 5 depicts an embodiment of the present invention placed on a spine.

FIG. 6 depicts an embodiment of the present invention placed on a spine.

FIG. 7 depicts an embodiment of the present invention placed on a spine.

FIG. 8 is a perspective view of a bone contacting extension according to an embodiment of the present invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims. For illustrative purposes, cross-hatching, dashing or shading in the figures is provided to demonstrate sealed portions and/or integrated regions or devices for the package.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

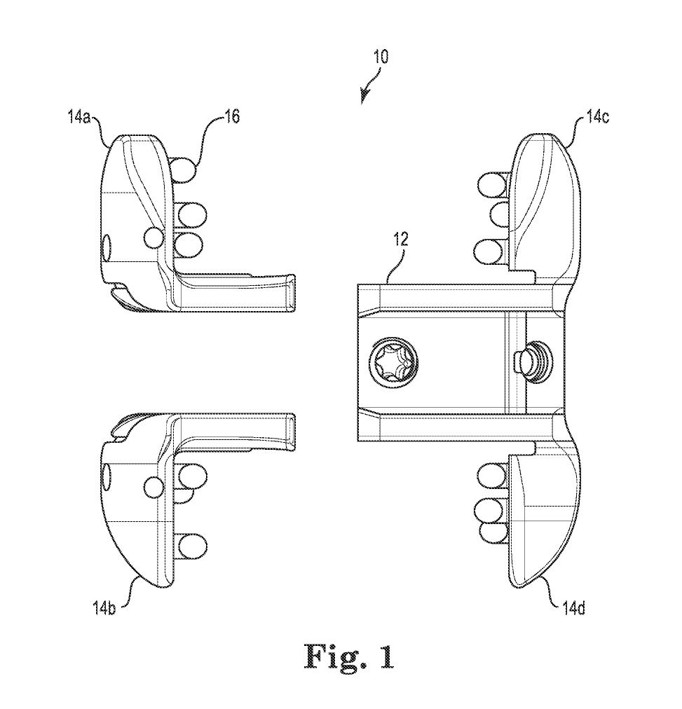

FIG. 1 depicts an embodiment of spacer 10 according to certain aspects of the present invention. Spacer 10 may include spacer body 12. Spacer body 12 may have a width in the range of about 8-20 millimeters. Bone contacting extensions 14a-14d may be movably connected to spacer body 12.

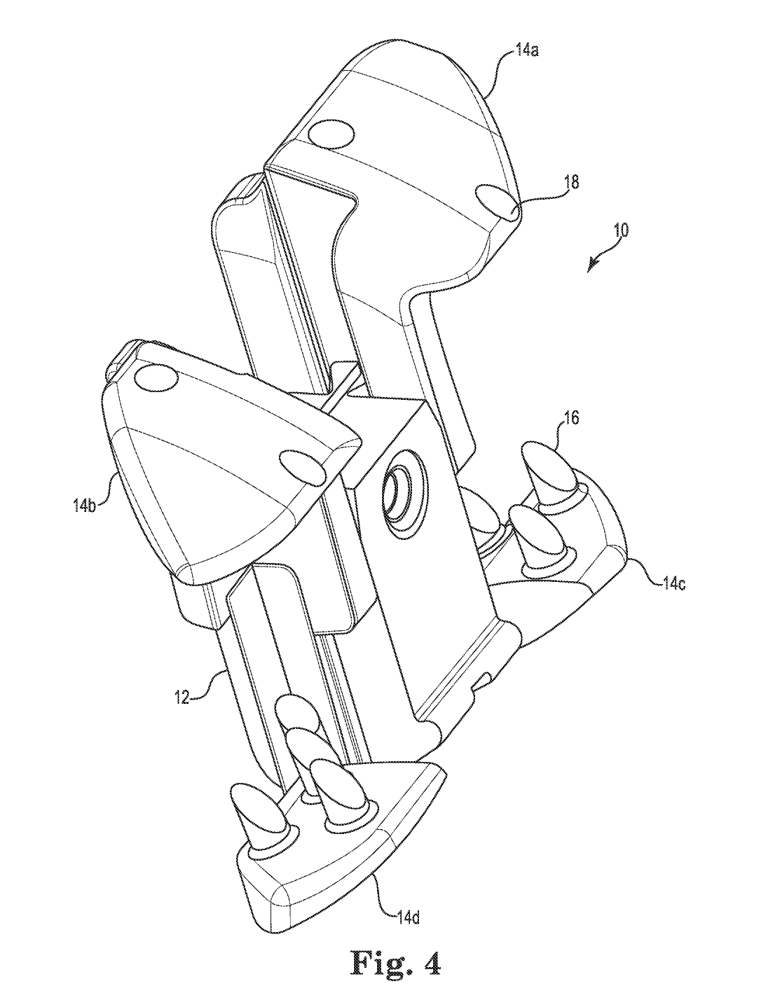

Extensions 14a-14d may move independently of each other to accommodate variations in patient anatomy. Bone engaging projections 16 may be seen in FIGS. 1-4. Projections 16 may be angled down toward the bone to assist in pulling the implant tightly against the bone.

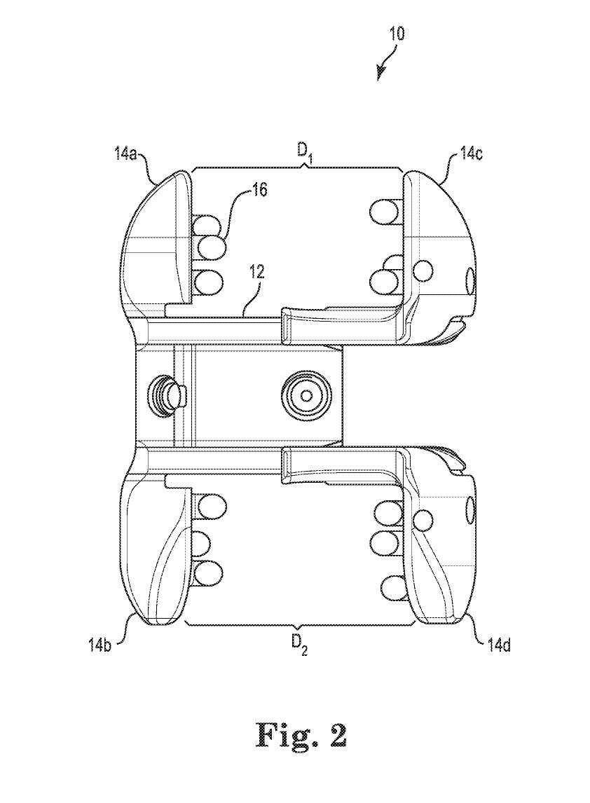

Spacer 10 may be inserted between a patient's spinous process as a one piece construct. As a one piece construct, as shown in FIG. 2, spacer 10 may be placed down over the spinous process and rotated into the desired position. Once inserted, a compression tool may connect to dimples 18 and the tool may compress spacer 10, pushing projections 16 into the bone. Spacer 10 may then be tightened into position.

In another embodiment, spacer 10 may be assembled in situ. In such an embodiment, extensions 14 may be mounted on a distraction tool and placed on the interspinous process. The distraction tool may then be used to distract the interspinous process space. The distraction tool may incrementally move bone contacting extensions 14a-d, thus opening spacer 10 to produce incremental distraction. Once the desired distraction is accomplished, the physician may perform a decompression. Spacer body 12 may then be placed onto the extensions, completing the construct. A compression tool may connect to dimples 18 and the tool may compress spacer 10, pushing projections 16 into the bone. Spacer 10 may then be tightened into position.

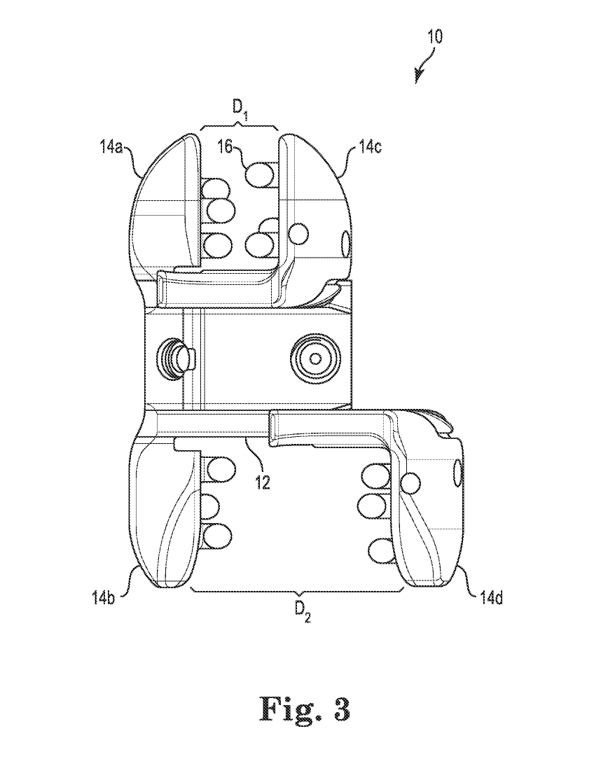

As can be seen in FIG. 2, extensions 14a-d may be incrementally moved to create a distance, D1, between 14a and 14c and/or D2 between extensions 14b and 14d. Distances, D1 and D2 may be in the range of about 3-18 millimeters. Distances D1 and D2 are independent of each other, thus spacer 10 may be custom fit to the patient's anatomy. FIG. 3 depicts a smaller D1 and a relatively wider D2.

As can be seen in FIGS. 1-4, the interior aspect of extensions 14a-d is contoured to fit down very close to the patient's lamina. As is also shown in FIGS. 1-4, the exterior aspect of extensions 14a-d is shaped to such that one or more spacers may be placed very close together, allowing treatment of multiple spinal levels. The contouring of extensions 14a-d further permits use of the spacer at L5-S1.

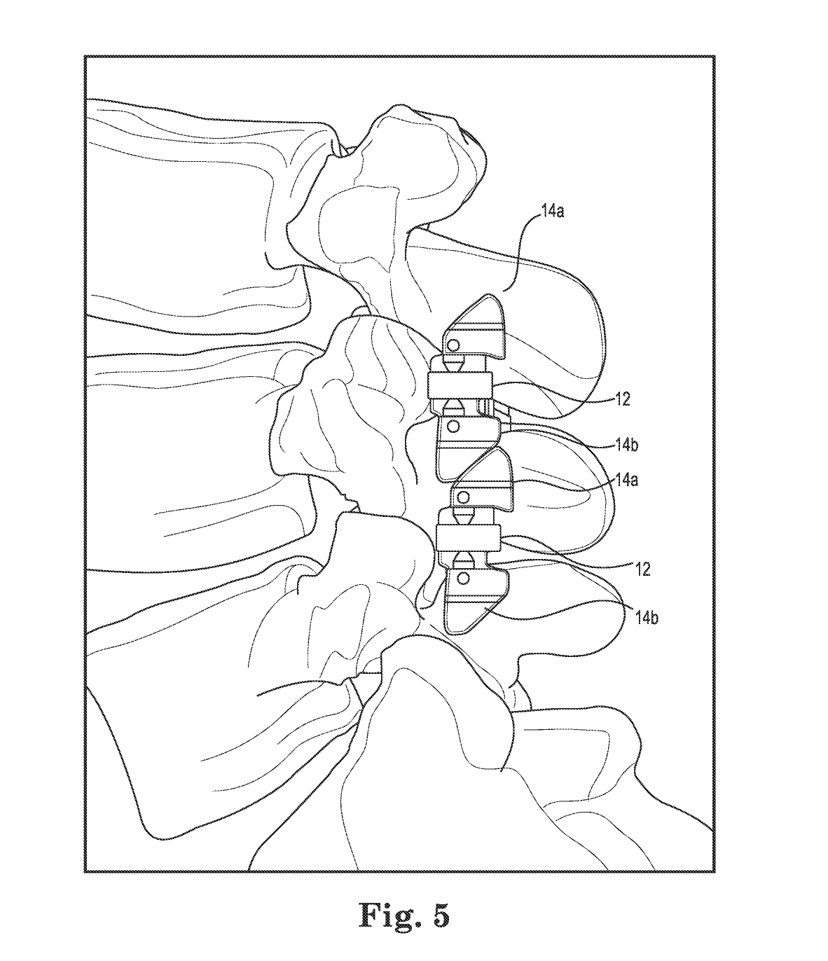

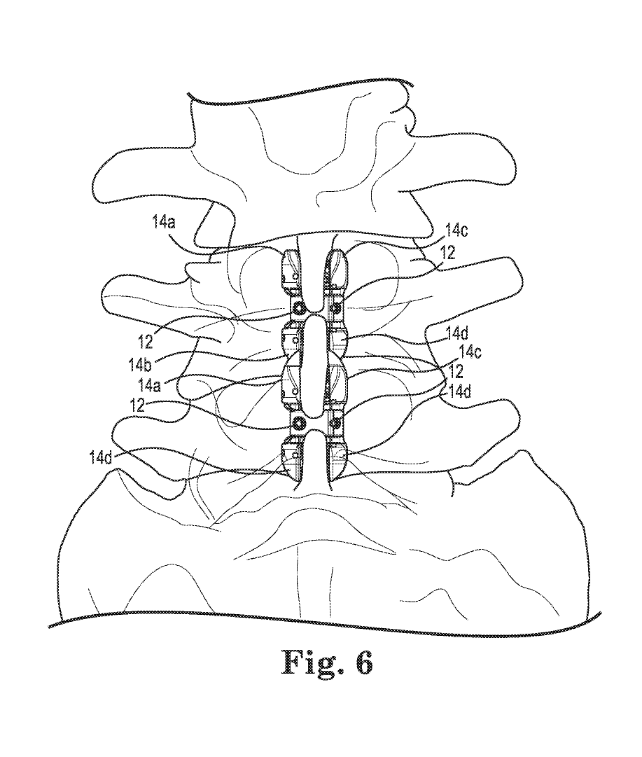

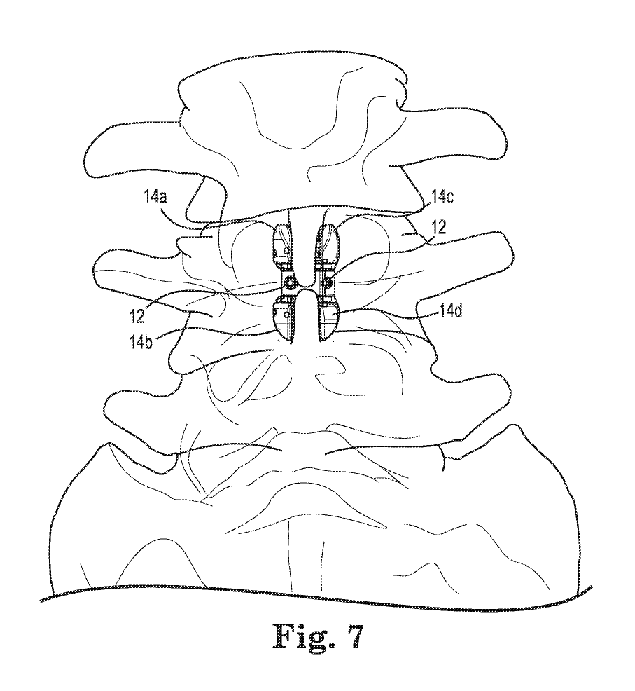

FIGS. 5 and 6 depict how the exterior shape of extensions 14a-14d allows two spacers according to the present invention to be placed tightly next to each other on a spine. FIG. 7 depicts a top view of a single spacer of the present invention placed on a spine. In an alternate embodiment, spacer 10 may be implanted in a desired open position and affixed to the patient.

In yet another embodiment, spacer 10 may be used to compress the interspinous process. In this embodiment, an anterior plate may be used with spacer 10. In this embodiment, the physician may choose a spacer narrow than the interspinous process space. A compression tool may connect to spacer 10 at dimples 18 and be used to compress the spacer and the interspinous process space.

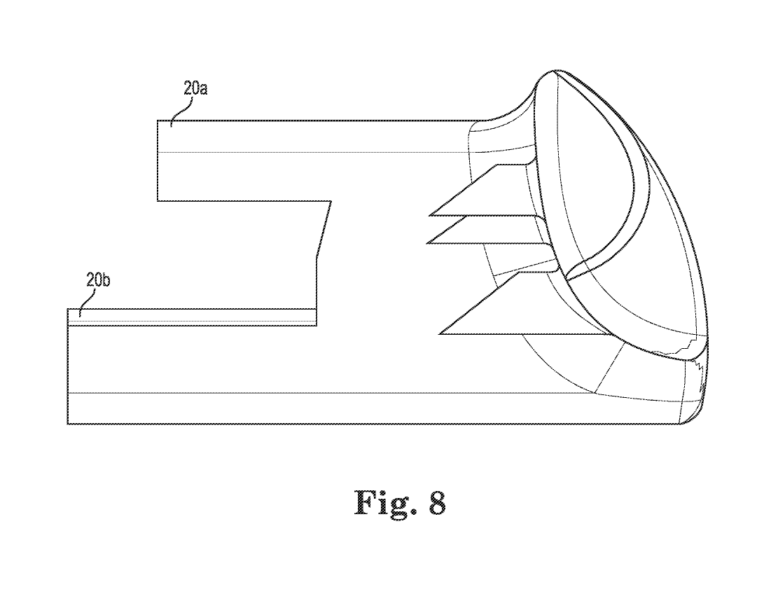

As can be seen in FIG. 8, bone contacting extensions may have arms 20a and 20b of different lengths. The example embodiment depicted in FIG. 8 includes arm 20b having a longer length that arm 20a. The extra length on arm 20b may provide a surface contact point for guidance as the implant is being assembled in situ.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. An interspinous process spacer comprising:
a spacer body having bone contacting extensions movably connected thereto.
